(19) United States
(12) Patent Application Publication
(10) Pub. No.: US 2013/0202682 A1
(43) Pub. Date: Aug. 8, 2013

(54) SYNTHETIC MATRIX VESICLES FOR BIOMIMETIC MINERALIZATION

(71) Applicant: The Trustees of Columbia University in the City of New York, New York, NY (US)

(72) Inventors: Helen H. Lu, New York, NY (US); Philip Chuang, Glastonbury, CT (US)

(73) Assignee: The Trustees of Columbia University in the City of New York, New York, NY (US)

(21) Appl. No.: 13/749,122

(22) Filed: Jan. 24, 2013

Related U.S. Application Data
(60) Provisional application No. 61/590,028, filed on Jan. 24, 2012.

(51) Int. Cl. A61K 38/43; A61K 38/18; A61K 33/42; A61K 45/06; A61K 9/127; A61K 33/06

(52) U.S. Cl. CPC A61K 38/43; A61K 9/127; A61K 33/06; A61K 33/42; A61K 45/06; A61K 38/18
USPC 424/450; 424/490; 424/696; 424/601; 435/375

(57) ABSTRACT

Compositions of synthetic matrix vesicles with mineralization relevant ions which direct mineralization of a biomimetic mineral phase and methods for use thereof are provided.

This patent application claims the benefit of priority from U.S. Provisional Application Ser. No. 61/590,028, filed Jan. 24, 2012, teachings of which are herein incorporated by reference in their entirety.

### FIELD

The disclosed subject matter relates to synthetic matrix vesicles which direct mineralization of a biomimetic mineral phase.

### BACKGROUND

Bone serves as the main structural unit of the human body and is the most commonly replaced organ. In the United States, 800,000 grafting procedures are performed annually. However, allografts and autografts are limited by lack of structural integrity, donor-site morbidity, poor bioactivity, failure to revascularize and/or mechanical mismatch. Natural or synthetic materials are used as biological cement, scaffolding and coating materials for implants to promote mineral deposition at the site of interest.

### SUMMARY

An aspect of the disclosed subject matter relates to a composition which directs mineralization of a biomimetic mineral phase. The compositions comprise a synthetic matrix vesicle encapsulating one or more mineralization relevant ions. In one embodiment, the synthetic matrix vesicle is a liposome.

Another aspect of the present invention relates to a method for promoting biosynthesis and/or mineralization potential of cells comprising treating the cells with a composition comprising a plurality of synthetic matrix vesicles encapsulating one or more mineralization relevant ions.

Another aspect of the disclosed subject matter relates to a method for facilitating biomimetic mineralization on, for example, tissue engineering scaffolds or dentin or enamel surfaces, via a composition comprising a plurality of the synthetic matrix vesicles encapsulating one or more mineralization relevant ions. In one embodiment, the synthetic matrix vesicles comprise liposomes.

Another aspect of the disclosed subject matter relates to a method for improving biological fixation of a graft and/or scaffold at a repair site, said method comprising adding a composition comprising a plurality of synthetic matrix vesicles encapsulating one or more mineralization relevant ions to the graft, scaffold and/or repair site. In one embodiment, the synthetic matrix vesicles comprise liposomes.

Yet another aspect of the disclosed subject matter relates to devices which promote mineralization comprising a composition of synthetic matrix vesicles encapsulating one or more mineralization relevant ions and collagen gel or collagen nanofibers and methods for use of these devices in promoting mineralization.

### BRIEF DESCRIPTION OF THE FIGURES

FIGS. 1A-1C provide schematics of various synthetic matrix vesicles encapsulating the mineralization relevant ion Ca²⁺ (FIG. 1A), the mineralization relevant ion Pi (FIG. 1B) and the mineralization relevant ions Ca²⁺ and Pi.

FIGS. 2A-C show the average diameter of a liposomal embodiment of synthetic matrix vesicles encapsulating the mineralization relevant ion Ca²⁺ and the mineralization relevant ion Pi determined by cryo-EM (FIGS. 2A and 2B, respectively) and by zetasizer (FIG. 2C).

FIG. 3A-3B are bargraphs displaying calcium (FIG. 3A) and (FIG. 3B) phosphate ion concentrations detected in the synthetic matrix vesicles.

FIG. 4A-4C show the effects of modifying the ratio of 1,2-dipalmitoyl-sn-glycero-3-phosphocholine (DPPC) to 1,2-bis(myristoyl)-sn-glycero-3-phosphocholine (DMPC) in the lipid membrane of the synthetic matrix vesicle (FIG. 4A) on transport of calcium ions (FIG. 4B) and phosphate ions (FIG. 4C) across the synthetic matrix vesicle lipid membrane.

FIG. 5 is a bargraph displaying incorporation alkaline phosphatase internally and/or externally into the synthetic matrix vesicle.

FIGS. 6A and 6B are bargraphs comparing osteoblast cell growth (FIG. 6A) and mineralization potential based upon alkaline phosphatase activity (ALP) activity (FIG. 6B) in cells treated with equivalent doses of synthetic matrix vesicles of liposomes with no mineralizing ions (Cont Lip), calcium ions (Ca Lip), phosphate ions (P Lip) or a mixture thereof (CaP Lip).

FIGS. 7A and 7B are bargraphs comparing osteoblast cell growth (FIG. 7A) and mineralization potential based upon alkaline phosphatase activity (ALP) activity (FIG. 7B) in cells treated with different concentrations of synthetic matrix vesicles of liposomes with phosphate ions (P Lip) with (FIG. 7B) and without (FIG. 7A).

FIGS. 8A and 8B are bargraphs showing results of additional experiments comparing osteoblast cell growth (FIG. 8A) and mineralization potential based upon alkaline phosphatase activity (ALP) activity (FIG. 8B) in cells treated with different concentrations of synthetic matrix vesicles of liposomes with phosphate ions (P Lip) with (FIG. 8B) and without (FIG. 8A).

FIG. 9 provides a schematic of incorporation of synthetic matrix vesicles into a nanofiber scaffold.

### DETAILED DESCRIPTION

### Definitions

In order to facilitate an understanding of the material which follows, one may refer to Freshney, R. Ian. Culture of Animal Cells—A Manual of Basic Technique (New York: Wiley-Liss, 2000) for certain frequently occurring methodologies and/or terms which are described therein.

Unless defined otherwise, all technical and scientific terms used herein have the meaning commonly understood by a person skilled in the art to which this invention belongs. However, except as otherwise expressly provided herein, each of the following terms, as used in this application, shall have the meaning set forth below.

As used herein, “biomimetic” shall mean a resemblance of a synthesized material to a substance that occurs naturally in a human body and which is not substantially rejected by (e.g., does not cause an unacceptable adverse reaction in) the human body. When used in connection with the tissue scaffolds, biomimetic means that the scaffold is substantially biologically inert (i.e., will not cause an unacceptable immune response/rejection) and is designed to resemble a structure (e.g., soft tissue anatomy) that occurs naturally in a mammalian, e.g., human, body and that promotes healing when implanted into the body.

As used herein, “microfiber” shall mean a fiber with a diameter no more than 1000 micrometers.

As used herein, “nanofiber” shall mean a fiber with a diameter no more than 1000 nanometers.

In one embodiment, the microfibers and/or or nanofibers are comprised of a biodegradable polymer that is electrospun into a fiber. The microfibers and/or nanofibers of the scaffold are oriented in such a way (i.e., aligned or unaligned) so as to mimic the natural architecture of the soft tissue to be repaired. Moreover, the microfibers and/or nanofibers and the subsequently formed microfiber and/or nanofiber scaffold are controlled with respect to their physical properties, such as for example, fiber diameter, pore diameter, and porosity so that the mechanical properties of the microfibers and/or nanofibers and microfiber and/or nanofiber scaffold are similar to the native tissue to be repaired, augmented or replaced.

As used herein, “osteoblast” shall mean a bone-forming cell which may be derived from mesenchymal osteoprogenitor cells and which forms an osseous matrix in which it becomes enclosed as an osteocyte. The term may also be used broadly to encompass osteoblast-like, and related, cells, such as osteocytes and osteoclasts. An “osteoblast-like cell” means a cell that shares certain characteristics with an osteoblast (such as expression of certain proteins unique to bones), but is not an osteoblast. “Osteoblast-like cells” include preosteoblasts and osteoprogenitor cells.

As used herein, “osteogenesis” shall mean the production of bone tissue.

As used herein, “osteointegrative” means having the ability to chemically bond to bone.

As used herein, “stem cell” means any unspecialized cell that has the potential to develop into many different cell types in the body, such as mesenchymal osteoprogenitor cells, osteoblasts, osteocytes, osteoclasts, chondrocytes, chondrocyte progenitor cells, fibrochondrocytes, fibroblasts and fibroblast progenitor cells. Nonlimiting examples of “stem cells” include mesenchymal stem cells, embryonic stem cells and induced pluripotent cells.

As used herein, “synthetic” shall mean that the material is not of a human or animal origin.

As used herein, all numerical ranges provided are intended to expressly include at least the endpoints and all numbers that fall between the endpoints of ranges.

The following embodiments are provided to further illustrate the methods of tissue scaffold production of this application. These embodiments are illustrative only and are not intended to limit the scope of this application in any way.

### Embodiments

In the field of orthopedic and dental surgical procedures, natural or synthetic materials are oftentimes used as biological cement, scaffolding and coating materials for implants to promote mineral deposition at the site of interest. However, limited time access to the site requires use of large concentrations of these materials which is non-physiologic and can have cytotoxic as well as immunogenic detrimental side effects.

The disclosed subject matter provides compositions and methods for cell-mediated mineral deposition at the site of interest, thereby avoiding the possibility of exposure to non-physiologic concentrations and possible detrimental side effects.

The disclosed subject matter relates to compositions of synthetic matrix vesicles and methods for directing mineralization of a biomimetic mineral phase via compositions of synthetic matrix vesicles. The compositions and methods are useful in biological fixation of a graft and/or scaffold at a repair site as such compositions function as biological cement, scaffolding and/or implant coatings.

In one embodiment, the synthetic matrix vesicles of the compositions comprise liposomes. In one embodiment, the liposomes comprise 1,2-dipalmitoyl-sn-glycero-3-phosphocholine (DPPC). In one embodiment, the liposomes comprise or 1,2-bis(myristoyl)-sn-glycero-3-phosphocholine (DMPC). In one embodiment, the liposomes comprise DPPC and DMPC. It is expected, however, that other components known for use in formation of liposomes can also be used in the synthetic matrix vesicles.

In another embodiment the synthetic matrix vesicle is comprised of another material such as block-copolymer micelles.

In one embodiment, the synthetic matrix vesicles range in diameter size from about 200 nm to about 300 nm to mimic the size of native matrix vesicles.

In one embodiment, the synthetic matrix vesicles of the composition encapsulate one or more mineralization relevant ions. Examples include, but are not limited to, Ca²⁺ and Pi. Other dissolvable ion sources, such as, but in no way limited to beta-glycerophosphate, can also be encapsulated, as well as carbonates for different types of mineral formation. Compositions may include synthetic matrix vesicles encapsulating the same mineralization relevant ions or different mineralization relevant ions. For example, in one embodiment, the composition may comprise synthetic matrix vesicles encapsulating Ca2+. In one embodiment, the composition may comprise synthetic matrix vesicles encapsulating Pi. In one embodiment, the composition may comprise synthetic matrix vesicles encapsulating Ca2+ and synthetic matrix vesicles encapsulating Pi.

The synthetic matrix vesicles of the composition may also encapsulate other ions as well as active agents such as, but not limited to, antibiotics, growth factors, ionophores, enzymes, and calcium ion channels such as, but not limited to, Annexin V, as well as phosphate ion channels.

In one embodiment, the composition may comprise an alternative or additional agent which promotes mineralization in the presence of organic phosphates such as alkaline phosphatase incorporated internally within the synthetic matrix vesicles in addition to a mineralization relevant ion such as Ca2+ or Pi and/or externally attached to the synthetic matrix vesicles. In one embodiment, the compositions comprises synthetic matrix vesicles with alkaline phosphatase attached or conjugated externally to the synthetic matrix vesicles.

In one embodiment, compositions of this disclosure are formulated to be injectable.

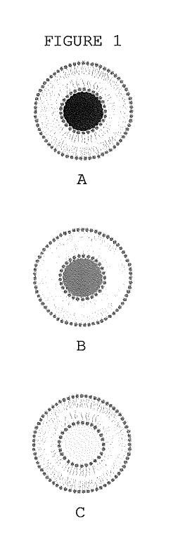

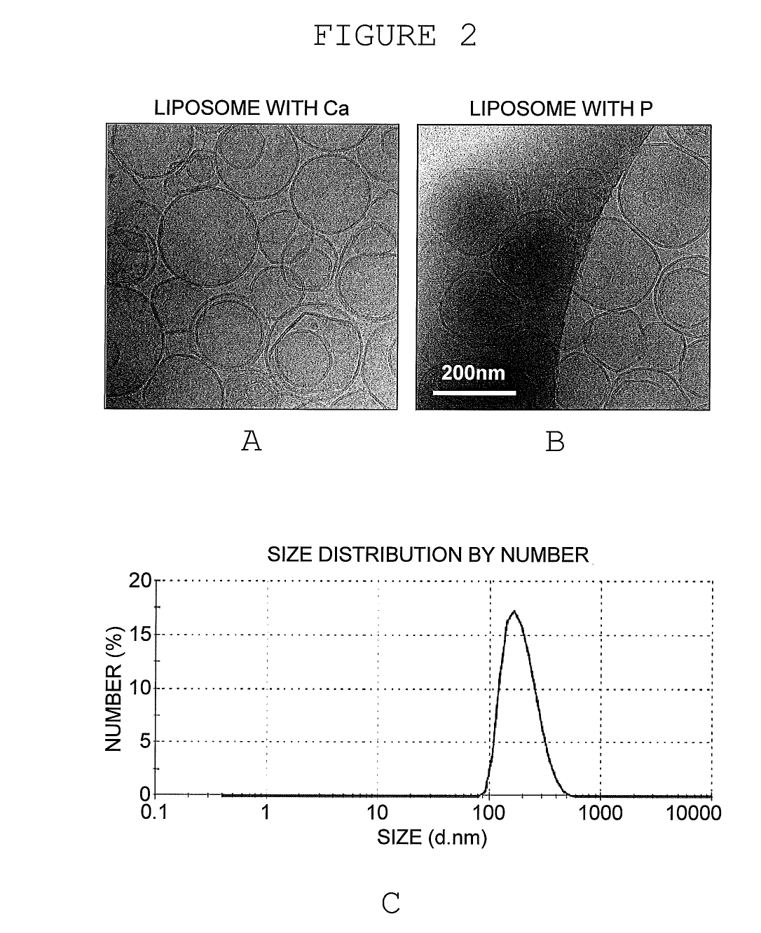

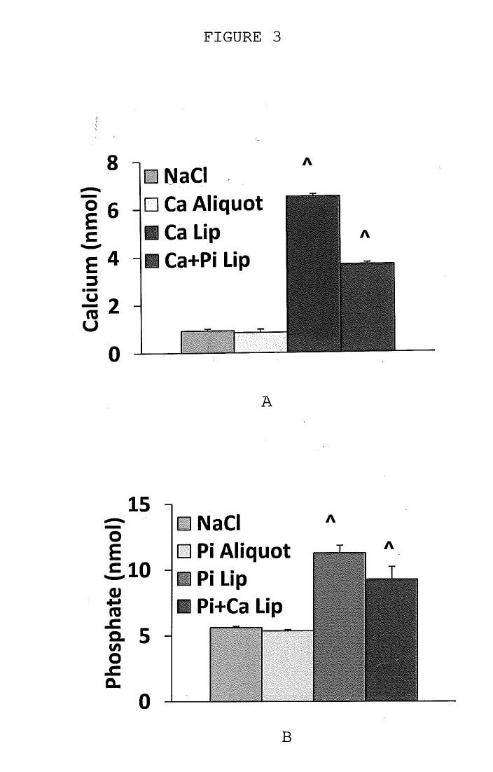

Compositions of synthetic matrix vesicles comprised of liposomes formed from DPPC, DMPC, or DPPC and DMPC encapsulating mineralization relevant ions such as Ca²⁺ and Pi were prepared. See FIG. 1. The average diameter of the liposomes in the compositions were 215±6 nm confirmed by cryo-EM and zetasizer. See FIG. 2. As shown in FIG. 3, calcium and phosphate ions were detectable in the synthetic matrix vesicles.

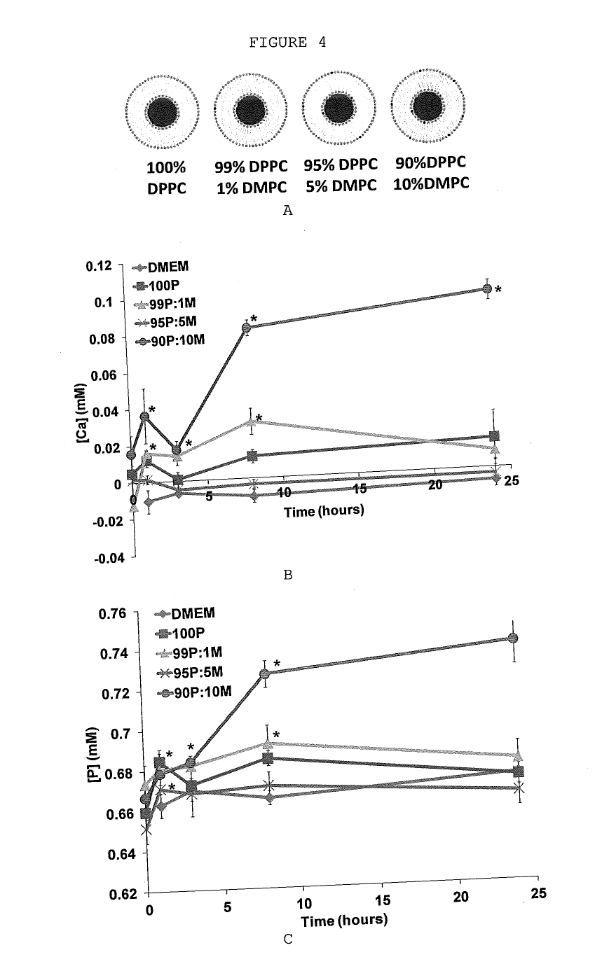

Further, as shown in FIG. 4, the phospholipid membrane can be modified to control ion transport. Synthetic matrix vesicles with membranes made up of 99% DPPC and 90% DPPC showed a significant (p<0.05) increase in calcium ion release by 1 and 8 hours. Synthetic matrix vesicles with membranes made up of 90% DPPC showed a further significant (p<0.05) increase in calcium ion release by 24 hours. A significant (p<0.05) increase in phosphate ion release occurred by 1 hour in synthetic matrix vesicles with membranes made up of 100% DPPC and 95% DPPC and by 8 hours in synthetic matrix vesicles with membranes made up of 99% DPPC and 90% DPPC.

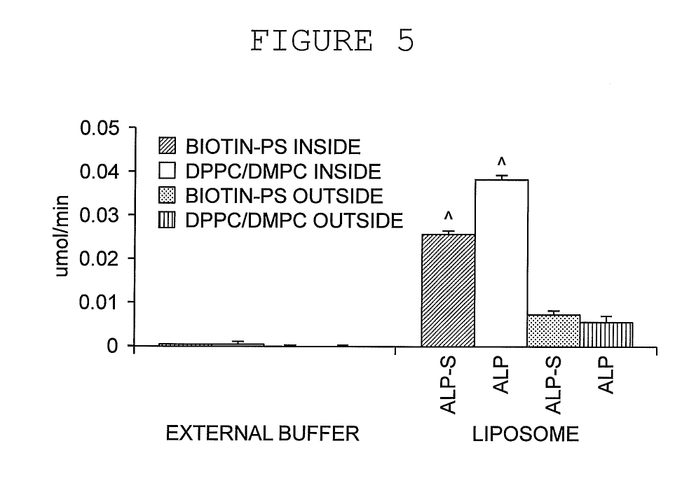

The synthetic matrix vesicles of the composition were further modified to be more biomimetic through internal incorporation as well as external conjugation via biotin of the additional mineralizing agent alkaline phosphatase. See FIG. 5.

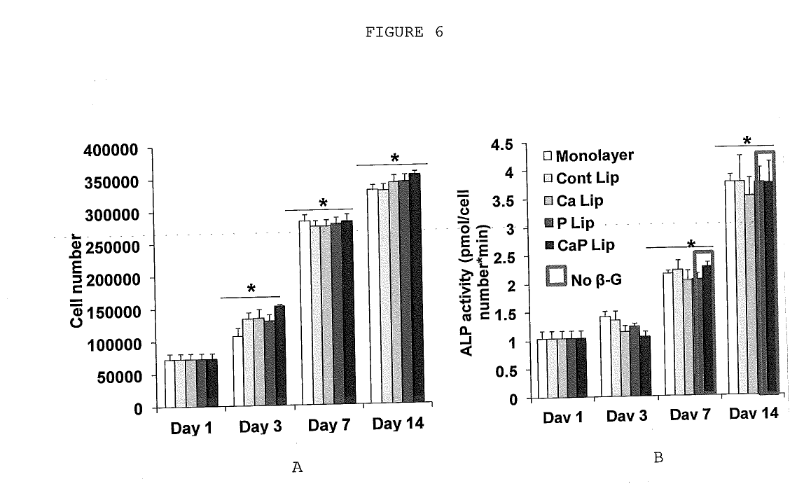

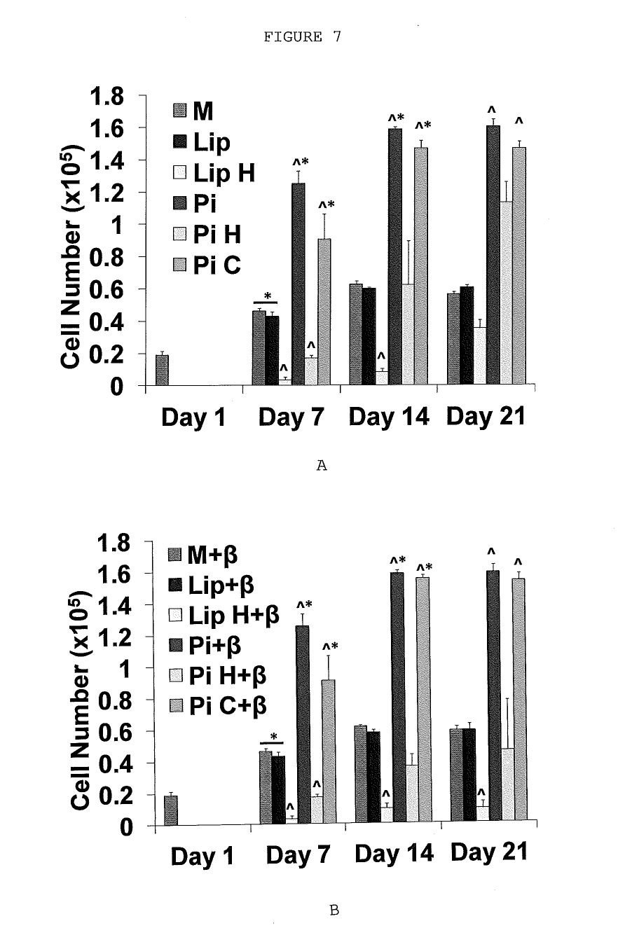

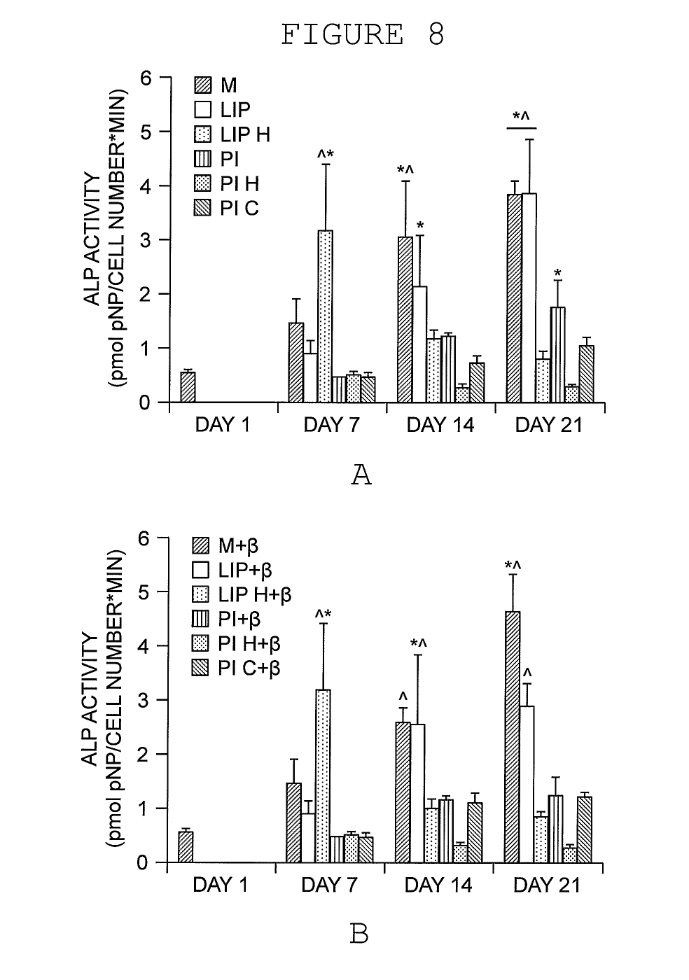

Cell growth (FIG. 6A) and mineralization potential based upon ALP activity (FIG. 6B) of osteoblast-like treated with equivalent doses of synthetic matrix vesicles of liposomes with no mineralizing ions (Cont Lip), calcium ions (Ca Lip), phosphate ions (P Lip) or a mixture thereof (CaP Lip) were also determined. Cell number increased significantly by days 3, 7 and 14 in all groups. See FIG. 6A. ALP activity increased by days 7 and 14 for all groups. Cells exposed to synthetic matrix vesicles of liposomes with phosphate ions showed similar levels of ALP activity to groups exposed to the osteogenic additive β-glycerophosphate on day 7. However, histologically, cultures exposed to β-glycerophosphate showed more mineral-like structures than cells exposed to synthetic matrix vesicles of liposomes with phosphate ions. Further experiments with synthetic matrix vesicles of liposomes with phosphate ions at different doses were shown to affect mineralization response with and without β-glycerophosphate. See FIGS. 7 and 8.

Compositions disclosed herein are thus expected to be useful in a variety of mineralization applications.

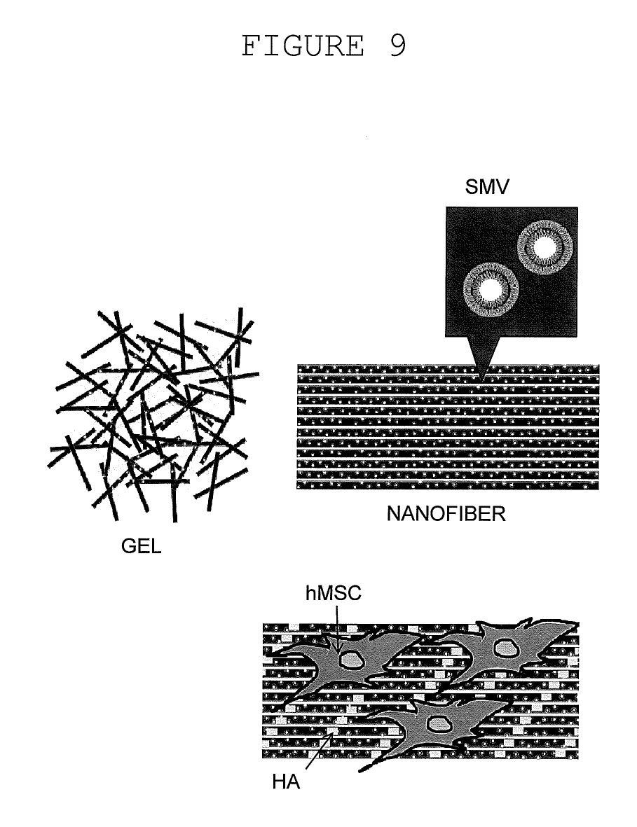

In one embodiment, the composition disclosed herein is incorporated into a tissue scaffold matrix such as a collagen based matrix to form a device which promotes mineralization. In one embodiment, the collagen based matrix is a gel matrix. In another embodiment, the collagen based matrix is a collagen nanofiber matrix of unaligned or aligned nanofibers. See FIG. 9. In these embodiment, the device may be injectable. In these embodiments, the tissue scaffold matrix incorporated with the composition of synthetic matrix vesicles may be seeded with cells. Examples of such cells include, but are not limited to, osteoblast-like cells and stem cells capable of differentiating to osteoblast-like cells. In this embodiment, the tissue scaffold matrix incorporated with synthetic matrix vesicles may further comprise one or more growth factors.

In one embodiment, the composition of synthetic matrix vesicles is used to promote biosynthesis and/or mineralization potential of cells comprising by treating the cells with the composition of synthetic matrix vesicles. In one embodiment, the cells treated are osteoblast-like cells. In one embodiment, the cells treated are stem cells capable of differentiating to osteoblast-like cells. In one embodiment, the synthetic matrix vesicles further comprise alkaline phosphatase conjugated to the synthetic matrix vesicles. In this embodiment, an organic phosphate may be externally added to further promote mineralization.

In one embodiment, the composition of synthetic matrix vesicles is used to facilitate biomimetic mineralization on, for example, tissue engineering scaffolds or dentin or enamel surfaces. In this embodiment, the composition is coated on the scaffold, dentin or enamel surface to promote and/or enhance mineralization.

In one embodiment a plurality of the synthetic matrix vesicles is used to improve biological fixation of a graft and/or scaffold at a repair site.

The disclosed subject matter is further illustrated by the following nonlimiting examples.

### EXAMPLES

### Example 1

### Liposome Synthesis

A thin film of DPPC/DPMC at the desired ratio was formed by chloroform evaporation in a round bottom flask. 0.5 ml of 0.2M ion solution per 2.5 mg of lipid is ten added at 60° C. The resulting solution was subjected to a freeze-thaw 3 times to promote homogenous ions inside and outside the liposomes. The liposome/ion solution was then passed through 200 nm pore extruder at 60° C. 20 times, washed three times, and suspended in 0.285M NaCl to remove unencapsulated ions.

### Example 2

### Cell Culture and Experimental Design

Cells used in all experiments were human osteoblast like cells (Saos-2). Monolayers were seeded at a density of 50,000 cells/cm². Cells were treated with 100 μg of control 90% DPPC/10% DMPC liposomes, Ca ion encapsulated liposomes, Pᵢ ion encapsulated liposomes, and a mixture of Ca and Pᵢ (ratio 1.67) ion encapsulated liposomes 24 hours after initial seeding. Cultures were maintained in fully supplemented media with 50 μg/ml of ascorbic acid through day 14. β-glycerophosphate was added on day 7 to groups without Pᵢ ion encapsulated liposomes. For experiments presented in FIGS. 7 and 8, half of all groups were treated with beta-glycerophosphate, and half were not. The dosage for the Pi liposomes were 1 mg/ml of Pi liposomes made with 0.2M NaPO4(3−)(Pi), 5 mg/ml of Pi liposomes made with 0.2M NaPO4(3−) (PiH) and 1 mg/ml of Pi liposomes made with 1.0M NaPO4(3−) (PiC).

### Example 3

### End Point Analyses

Liposomes were visualized using cryo-electron microscopy (Tecnai F20; 200 kv accelerating voltage). Size distribution was measured using a Malvern Zetasizer Nano-ZS (n=5). Ion concentration (n=5) was quantified using the arsenazo III modified calcium assay (Pointe Scientific) and a malachite green, ammonium molybdate based phosphate colorimetric assay (BioVision). Cell proliferation (n=5) was evaluated using the Picogreen dsDNA assay (Molecular Probes). Mineralization potential (n=5) was detected using a colorimetric assay for alkaline phosphatase (ALP) and von Kossa stain was used to evaluate mineral distribution on day 21 (n=2).

### Example 4

### Statistical Analysis

Multi-way ANOVA and the Tukey-HSD post-hoc test was used for all pair-wise comparisons (p<0.05).

The following disclosure should not be construed as limiting the invention in any way. One of skill in the art will appreciate that numerous modifications, combinations, rearrangements, etc. are possible without exceeding the scope of the invention. While this invention has been described with an emphasis upon various embodiments, it will be understood by those of ordinary skill in the art that variations of the disclosed embodiments can be used, and that it is intended that the invention can be practiced otherwise than as specifically described herein.

What is claimed is:

1. A composition which directs mineralization of a biomimetic mineral phase, said composition comprising synthetic matrix vesicles encapsulating one or more mineralization relevant ions.

2. The composition of claim 1 wherein the synthetic matrix vesicles are liposomes.

3. The composition of claim 2 wherein the liposomes are formed from 1,2-dipalmitoyl-sn-glycero-3-phosphocholine (DPPC).

4. The composition of claim 2 wherein the liposomes are formed from 1,2-bis(myristoyl)-sn-glycero-3-phosphocholine (DMPC).

5. The composition of claim 2 wherein the liposomes comprise DPPC and DMPC.

6. The composition of claim 1 wherein the synthetic matrix vesicles are block-copolymer micelles.

7. The composition of claim 1 wherein the one or more mineralization relevant ions are selected from the group consisting of Ca²⁺, Pi and mixtures thereof.

8. The composition of claim 1 comprising first synthetic matrix vesicles encapsulating one or more mineralization relevant ions and second synthetic matrix vesicles encapsulating one or more different mineralization relevant ions.

9. The composition of claim 1 further comprising an additional agent to promote mineralization incorporated internally within the synthetic matrix vesicles.

10. The composition of claim 1 further comprising an additional agent to promote mineralization incorporated externally on the synthetic matrix vesicles.

11. The composition of claim 1 further comprising an active agent encapsulated in the synthetic matrix vesicles.

12. The composition of claim 11 wherein the active agent is an antibiotic, growth factor, ionophore, enzyme, calcium ion channel or phosphate ion channel.

13. The composition of claim 1 which is injectable.

14. A method for promoting biosynthesis and/or mineralization potential of cells comprising treating the cells with the composition of claim 1.

15. A method for facilitating biomimetic mineralization on a tissue engineering scaffolds or dentin or enamel surfaces, said method comprising coating the scaffold or dentin or enamel surface with the composition of claim 1.

16. A method for improving biological fixation of a graft and/or scaffold at a repair site, said method comprising adding to the graft, scaffold and/or repair site the composition of claim 1.

17. A composition which directs mineralization of a biomimetic mineral phase, said composition comprising:
(a) synthetic matrix vesicles; and
(b) alkaline phosphatase encapsulated within or attached externally to the synthetic matrix vesicles.

18. The composition of claim 17 further comprising one or more mineralization relevant ions encapsulated in the synthetic matrix vesicles.

19. A device to promote mineralization, said device comprising the composition of claim 1 and collagen gel or collagen nanofibers.

20. A method for promoting biosynthesis and/or mineralization potential of cells comprising administering to the cells the device of claim 19.

21. The method of claim 20 wherein the device is administered by injection.

22. A device to promote mineralization, said device comprising the composition of claim 17 and collagen gel or collagen nanofibers.

23. A method for promoting biosynthesis and/or mineralization potential of cells comprising administering to the cells the device of claim 22.

24. The method of claim 23 wherein the device is administered by injection.
